(19) United States
(12) Patent Application Publication
(10) Pub. No.: US 2026/0144494 A1
(43) Pub. Date: May 28, 2026

(54) APPARATUS AND METHOD FOR DETERMINING QUALITY OF BIOSIGNAL DATA

(71) Applicant: SEOUL NATIONAL UNIVERSITY HOSPITAL, Seoul (KR)

(72) Inventor: Hyoung Woo CHANG, Seoul (KR)

(21) Appl. No.: 18/881,296

(22) PCT Filed: Jul. 7, 2023
(86) PCT No.: PCT/KR2023/009672

§ 371 Date: Nov. 13, 2025

(30) Foreign Application Priority Data
Jul. 7, 2022 (KR) 10-2022-0084021

(51) Int. Cl. A61B 5/00; A61B 5/346

(52) U.S. Cl. CPC A61B 5/7221; A61B 5/346; A61B 5/7257; A61B 5/7267

(57) ABSTRACT

Disclosed are an apparatus and method for determining the quality of biosignal data. The apparatus for determining the quality of biosignal data, according to an embodiment, divides a monitored biosignal into a plurality of segments of preset time units, classifies the plurality of segments into usable signals and unclear signals or unusable signals by using a pre-learned model including a first classification model and a second classification model, wherein the unclear signals or unusable signals are classified for remaining segments except for segments classified as the usable signals among the plurality of segments, and classifies, by using the pre-learned model, one or more segments that are classified as the unclear signals or unusable signals into the unclear signals and the unusable signals.

### DESCRIPTION OF STATE-SPONSORED RESEARCH AND DEVELOPMENT

This study was supported by the Ministry of Health and Welfare under [Project Name: Development of a Non-Invasive Blood Pressure Rapid Change Warning System through Deep Learning Application to Biosignals, Project Unique Number: 1465035901, Detailed Project Number: HI21C1324 (MSRI 08-2021-0222)].

### CROSS-REFERENCES TO RELATED APPLICATIONS

This application claims priority to Korean Patent Application No. 10-2022-0084021 filed on Jul. 7, 2022, the entire contents of which are incorporated into this application.

### TECHNICAL FIELD

The disclosed embodiments relate to techniques for determining data quality of biosignal.

### BACKGROUND

Since 2015, global efforts have been made to apply artificial intelligence to real life, and remarkable growth has been achieved in various fields such as autonomous driving, computer vision, and voice recognition. However, in the medical field, there have been few advances that may be said to have brought about a breakthrough change in the clinician's clinic site in relation to artificial intelligence. Biosignals (e.g., electrocardiogram (ECG), photoplethysmography (PPG), arterial blood pressure waveform, etc.) measured to monitor the patient's condition in the intensive care unit all have the characteristic of being able to be dataized numerically, but despite the development of modern physiology, the interpretation of changes in the physical condition reflected by these measurements is limited.

When an artificial intelligence technique is applied to biosignals to develop a diagnostic auxiliary algorithm or device, the biggest obstacle is all other artificial intelligence algorithm development processes and preprocessing processes. However, the biosignals measured in the intensive care unit are mixed with sections having good data quality and sections having poor data quality for various reasons.

Here, the biosignal having good data quality may be a clean signal normally measured without various noises such as a baseline sway, a pacemaker signal, motion artifacts, an electromyogram (EMG), and instrumentation noise, as shown in FIGS. 1A and 1B, respectively.

On the other hand, the biosignal with poor data quality may be a biosignal including an uninterpretable region as shown in FIGS. 2 and 3. For example, FIG. 2 shows an electrocardiogram with poor data quality. Specifically, the top left of FIG. 2 is a signal whose accurate heartbeat time point is difficult to grasp due to severe baseline sway, and the data quality is poor. In bottom left of FIG. 2, because the accurate heartbeat time point is difficult to grasp due to severe high frequency noise, the data quality is poor. The top right of FIG. 2 includes a defect that affects the artificial intelligence algorithm due to the artificial pacemaker signal being recorded together. Also, the top right of FIG. 2 has poor data quality for reasons including high frequency noise. The bottom right of FIG. 2 is unable to produce effective cardiac contraction due to fatal arrhythmia (ventricular fibrillation), and it is inappropriate to attempt any algorithmic interpretation based on such a signal.

Referring to FIG. 3, a photoplethysmography with poor data quality is shown. Specifically, the top left of FIG. 3 is a signal with very severe baseline sway, and the data quality is poor. The bottom left of FIG. 3 shows a severe artifact, which makes it impossible to interpret the signal. In the top right of FIG. 3, there is a problem of regular downward spikes appearing due to interference from the artificial pacemaker or electrical device. In bottom right of FIG. 3, there is a problem that the amplitude of the pulse is relatively small while the baseline sway is very severe.

That is, due to many factors such as active or passive posture change of the patient, treatment for clinic, entrance and exit of the patient, and movement for examination, there are meaningless data sections in the intensive care unit continuous monitoring data that cannot be used for any algorithm development, and these sections must be excluded not only in the algorithm development but also in the performance confirmation step. For this reason, an algorithm for determining a meaningless data section in biosignal is necessary.

### DETAILED DESCRIPTION

### Technical Problems

The disclosed embodiments are directed to determine a data quality of biosignal.

### Technical Solution

A method for determining the quality of biosignal data, according to an embodiment, is performed by an apparatus for determining the quality of biosignal data comprising: one or more processors; and a memory storing one or more programs executed by the one or more processors, the method comprising: dividing a monitored biosignal into a plurality of segments of preset time units; classifying the plurality of segments into usable signals and unclear signals or unusable signals, respectively, by using a pre-learned model comprising a first classification model and a second classification model, wherein the unclear signals or unusable signals are classified for remaining segments except for segments classified as the usable signals among the plurality of segments; and classifying, by using the pre-learned model, one or more segments that are classified as the unclear signals or unusable signals into the unclear signals and the unusable signals.

The biosignal may include at least one of an electrocardiogram and a photoplethysmography, and the method for determining the quality of biosignal data may further include, when the biosignal is an electrocardiogram, processing a Fast Fourier Transform (FFT) on the electrocardiogram.

The first classification model may be learned to classify the segments of biosignal input to the first classification model into usable signals and unclear signals or unusable signals, based on learning data labeled with an usable class and an unusable class, and the second classification model may be learned to classify the segments for biosignal input to the second classification model into unclear signals and unusable signals, based on learning data labeled with an usable class, an unclear class, and an unusable class.

The first classification model may be learned to, when a first feature of the segments of biosignal input to the first classification model is equal to or greater than a first numerical value, classify the segment of input biosignal into the unclear signals or unusable signals, and, when it is less than the first numerical value, classify it into the usable signals, the second classification model may be learned to, when a first feature of the segments of biosignal input to the second classification model is equal to or greater than a second numerical value having a value higher than the first numerical value, classify the input biosignal into the unusable signals, and, when it is equal to or greater than the first numerical value and less than the second numerical value, classify it into the unclear signals, and, when it is less than the first numerical values, classify it into the usable signals, and first feature may include a proportion of abnormal records containing noise or signal interruption in the segments of input biosignal.

The first classification model may be learned to classify the segments of input biosignal into the usable signals and the unclear signals or unusable signals, based on second feature of the segments of biosignal input to the first classification model, the second classification model may be learned to classify the segments of input biosignal into the usable signals, the unclear signals, and the unusable signals, based on second feature of the segments of biosignal input to the second classification model, and second feature may include at least one of a slope between a maximum point and a minimum point in a time domain of input biosignal, an amplitude of a peak, a time interval between peaks, a number of peaks for each of sections of the segments of input biosignal, an amplitude of maximum amplitude peak for each of the sections, an amplitude of minimum amplitude peak for each of the sections, a maximum value of peak interval for each of the sections, a minimum value of peak interval for each of the sections, and spectral entropy for each of the sections.

The first classification model may be learned to classify the segments of input biosignal into the usable signals and the unclear signals or unusable signals, based on third feature of the segments of biosignal input to the first classification model, the second classification model may be learned to classify the segments of input biosignal into the usable signals, the unclear signals, and the unusable signals, based on third feature of the segments of biosignal input to the second classification model, and third feature may include at least one of an average, a standard deviation value, of each of second features, and a number of instances that deviate from the range defined by the average plus or minus the standard deviation.

The first classification model may be learned to classify the segments of the input biosignal into the usable signals and the unclear signals or unusable signals, based on fourth feature of the segments of biosignal input to the first classification model, the second classification model may be learned to classify the segments of input biosignal into the usable signals, the unclear signals, and the unusable signals, based on fourth feature of the segments of biosignal input to the second classification model, and fourth feature may be based on a maximum power frequency on a time domain for each section of segments of input biosignal by performing FFT processing on each of the sections.

The fourth feature may include at least one of a standard deviation obtained by calculating for each section an average of maximum frequency difference values for each sub-section of each of the sections, an average maximum power frequency for each of the sections, and an average and a standard deviation of a differences between a first maximum power frequency and a second maximum power frequency for each of the sections.

An apparatus for determining the quality of biosignal data, according to an embodiment, is apparatus for determining the quality of biosignal data comprising: one or more processors; and a memory storing one or more programs executed by the one or more processors, wherein the one or more processors: divide a monitored biosignal into a plurality of segments of preset time units; classify the plurality of segments into usable signals and unclear signals or unusable signals, respectively, by using a pre-learned model comprising a first classification model and a second classification model, wherein the unclear signals or unusable signals are classified for remaining segments except for segments classified as the usable signals among the plurality of segments, and classify, by using the pre-learned model, one or more segments that are classified as the unclear signals or unusable signals into the unclear signals and the unusable signals.

The biosignal may include at least one of an electrocardiogram and a photoplethysmography, and the one or more processors, when the biosignal is an electrocardiogram, may process a Fast Fourier Transform (FFT) on the electrocardiogram.

The first classification model may be learned to classify the segments of biosignal input to the first classification model into usable signals and unclear signals or unusable signals, based on learning data labeled with an usable class and an unusable class, and the second classification model may be learned to classify the segments for biosignal input to the second classification model into unclear signals and unusable signals, based on learning data labeled with an usable class, an unclear class, and an unusable class.

The first classification model may be learned to, when a first feature of the segments of biosignal input to the first classification model is equal to or greater than a first numerical value, classify the segment of input biosignal into the unclear signals or unusable signals, and, when it is less than the first numerical value, classify it into the usable signals, the second classification model may be learned to, when a first feature of the segments of biosignal input to the second classification model is equal to or greater than a second numerical value having a value higher than the first numerical value, classify the input biosignal into the unusable signals, and, when it is equal to or greater than the first numerical value and less than the second numerical value, classify it into the unclear signals, and, when it is less than the first numerical values, classify it into the usable signals, and first feature may include a noise or signal interruption in the segments of input biosignal.

The first classification model may be learned to classify the segments of input biosignal into the usable signals and the unclear signals or unusable signals, based on second feature of the segments of biosignal input to the first classification model, the second classification model may be learned to classify the segments of input biosignal into the usable signals, the unclear signals, and the unusable signals, based on second feature of the segments of biosignal input to the second classification model, and second feature may include at least one of a slope between a maximum point and a minimum point in a time domain of input biosignal, an amplitude of a peak, a time interval between peaks, a number of peaks for each of sections of the segments of input biosignal, an amplitude of maximum amplitude peak for each of the sections, an amplitude of minimum amplitude peak for each of the sections, a maximum value of peak interval for each of the sections, a minimum value of peak interval for each of the sections, and spectral entropy for each of the sections.

The first classification model may be learned to classify the segments of input biosignal into the usable signals and the unclear signals or unusable signals, based on third feature of the segments of biosignal input to the first classification model, the second classification model may be learned to classify the segments of input biosignal into the usable signals, the unclear signals, and the unusable signals, based on third feature of the segments of biosignal input to the second classification model, and third feature may include at least one of an average, a standard deviation value, of each of second features, and a number of instances that deviate from the range defined by the average plus or minus the standard deviation.

The first classification model may be learned to classify the segments of the input biosignal into the usable signals and the unclear signals or unusable signals, based on fourth feature of the segments of biosignal input to the first classification model, the second classification model may be learned to classify the segments of input biosignal into the usable signals, the unclear signals, and the unusable signals, based on fourth feature of the segments of biosignal input to the second classification model, and fourth feature may be based on a maximum power frequency on a time domain for each section of segments of input biosignal by performing FFT processing on each of the sections.

The fourth feature may include at least one of a standard deviation obtained by calculating for each section an average of maximum frequency difference values for each sub-section of each of the sections, an average maximum power frequency for each of the sections, and an average and a standard deviation of a differences between a first maximum power frequency and a second maximum power frequency for each of the sections.

### Advantageous Effects

The disclosed embodiments may provide an apparatus and method for automatically determining the quality of biosignal without human intervention.

The disclosed embodiments may provide an apparatus and method for determining the quality of biosignal with high accuracy.

### BRIEF DESCRIPTION OF THE DRAWINGS

FIG. 1A is electrocardiogram of an example measured normally.

FIG. 1B is photoplethysmography of an example measured normally.

FIG. 2 is electrocardiogram of an example including an abnormally measured portion.

FIG. 3 is photoplethysmography of an example including an abnormally measured portion.

FIG. 4 is a block diagram for illustrating an apparatus for determining the quality of biosignal according to an embodiment.

FIG. 5 is an exemplary diagram for illustrating an example in which a plurality of segments are generated.

FIG. 6 is a block diagram for illustrating an apparatus for determining the quality of biosignal according to an additional embodiment.

FIG. 7 is an exemplary diagram for illustrating an example of a pre-learned model.

FIG. 8 is an exemplary diagram for illustrating an example of a gradient among features used by a pre-learned model for learning.

FIG. 9 is an exemplary diagram for illustrating an example of spectral entropy among features used by a pre-learned model for learning.

FIG. 10 is an exemplary diagram for illustrating an example of a first derivative coefficient among features used by a pre-learned model for learning.

FIG. 11 is an exemplary diagram for illustrating an example of the number of peaks among features used by a pre-learned model for learning.

FIG. 12 is an exemplary diagram for illustrating an example of a peak amplitude among features used by a pre-learned model for learning.

FIG. 13 is an exemplary diagram for illustrating an example of a maximum amplitude of a peak for each section among features used by a pre-learned model for learning.

FIG. 14 is an exemplary diagram for illustrating an example of a minimum amplitude of a peak for each section among features used by a pre-learned model for learning.

FIG. 15 is an exemplary diagram for illustrating an example of a minimum value of a peak interval for each section among features used by a pre-learned model for learning.

FIG. 16 is an exemplary diagram for illustrating an example of a maximum value of a peak interval for each section among features used by a pre-learned model for learning.

FIG. 17 is an exemplary diagram for illustrating an example standard deviation obtained by calculating an average of maximum frequency difference values for each sub-section among features used by a pre-learned model for learning.

FIG. 18 is an exemplary diagram for illustrating an example average and standard deviation of a difference between a first maximum power frequency and a second maximum power frequency for each section among features used by a pre-learned model for learning.

FIG. 19 is an exemplary diagram for illustrating architecture of an example of a model.

FIG. 20 is a graph showing the performance of a model having the architecture of FIG. 19 learned using the features used in FIGS. 8-18.

FIG. 21 is a graph showing performance of a model having the architecture of another example.

FIG. 22 is a flowchart for illustrating a method for determining the quality of biosignal according to an embodiment.

FIG. 23 is a flowchart for illustrating a method for determining the quality of biosignal according to an additional embodiment.

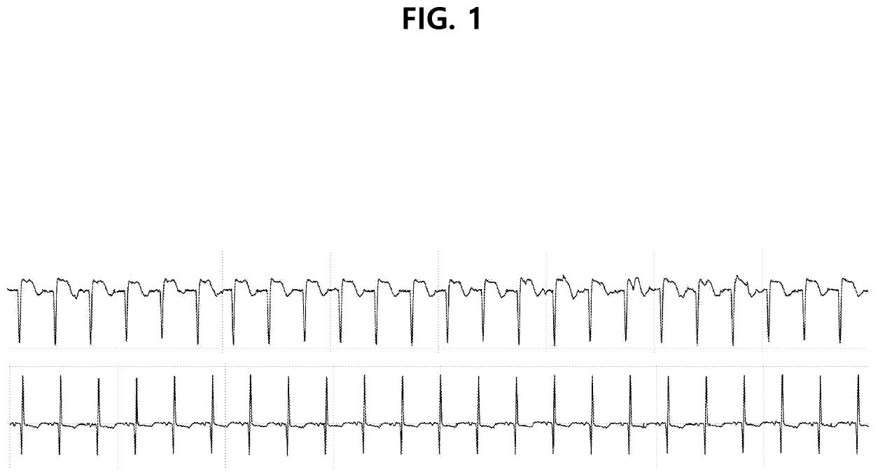

### EMBODIMENTS OF THE INVENTION

The terminology used herein is selected from general terms currently widely used as much as possible in consideration of functions, but may vary depending on the intention or practice of those skilled in the art, or the appearance of new technologies, etc. In addition, in a specific case, there is also a term arbitrarily selected by the applicant, and in this case, the meaning thereof will be described in that description section of the specification. Therefore, it should be understood that the terms used herein should be interpreted based on the substantial meaning of the terms and the content throughout the present specification, rather than the mere names of the terms.

The terms such as first, second are only used for the purpose of distinguishing one component from another. For example, a first component could be termed a second component, and, similarly, a second component could also be termed a first component, without departing from the scope of the present disclosure.

As used herein, the singular forms “a,” “an,” and “the” include plural referents unless the context clearly dictates otherwise. In this application, the terms such as “comprise” or “include”, “have” are intended to represent the presence of the component described herein or a combination thereof, but do not preclude the possibility that other components or features may be present or added.

Furthermore, embodiments described herein may have aspects that are wholly in hardware, partly in hardware and partly in software, or wholly in software. In this specification, a “unit”, a “module”, a “device”, a “server”, a “system”, or the like refers to a computer-related entity such as hardware, a combination of hardware and software, or software. For example, a unit, a module, a device, a server, or a system may refer to hardware constituting a part or all of a platform and/or software such as an application for driving the hardware.

The following describes the embodiments in detail with reference to the accompanying drawings and contents described in the accompanying drawings, but the scope to be claimed is not limited or restricted by the embodiments.

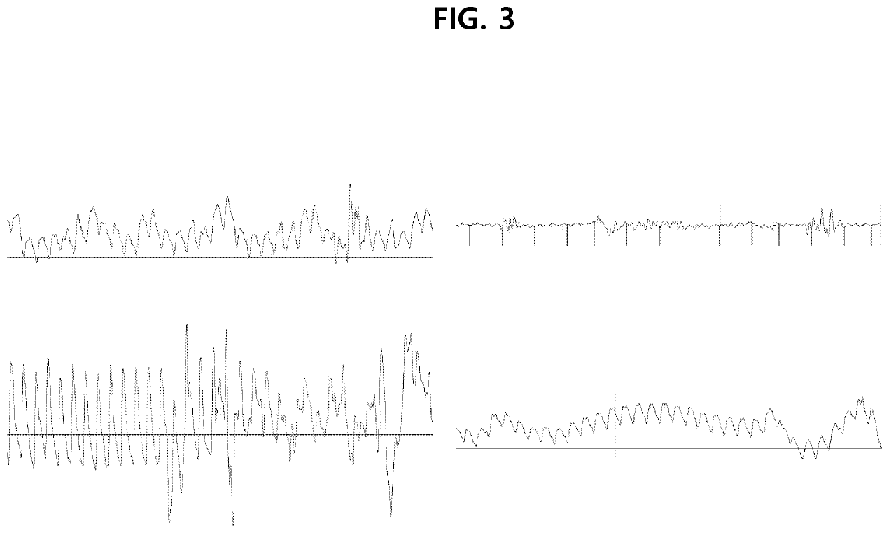

FIG. 4 is a block diagram for illustrating an apparatus 100 for determining the quality of biosignal according to an embodiment.

Referring to FIG. 4, the apparatus 100 for determining the quality of biosignal according to an embodiment includes a division unit 110, a first classification unit 120, and a second classification unit 130.

The division unit 110, the first classification unit 120, and the second classification unit 130 may be implemented using one or more physically separated devices, or may be implemented by one or more processors, or a combination of one or more processor and software, and may not be clearly distinguished in specific operations unlike the illustrated example.

The division unit 110 divides the monitored biosignal into a plurality of segments of preset time units. Here, the biosignal refers to a continuously occurring electrical signal between cells in the human body, and may include, for example, an electrocardiogram (ECG) and a photoplethysmography (PPG).

The division unit 110 may divide the entire monitored biosignal into a plurality of segments in a minimum unit time in which a human may determine quality at a glance. In this case, the unit time of the division unit 110 may mean a minimum delay time from real time.

Meanwhile, the target to be divided by the division unit 110 has been described as a biosignal, but this is an example, and the division unit 110 may divide the biosignal as well as each segment into a plurality of sections having a constant interval. In addition, the division unit 110 may divide each of the plurality of sections into a plurality of sub-sections having a constant interval. The target to be divided is not limited to the biosignal and the segment.

The first classification unit 120 classifies the plurality of segments into the usable signals and the unclear signals or unusable signals, respectively, by using a pre-learned model including the first classification model and the second classification model. Here, the unclear signals or the unusable signals mean the remaining segments among the plurality of segments except for the segments classified as the usable signals.

In particular, the first classification unit 120 may classify each of the plurality of segments into two classes (the “usable signal” and the “unclear signal or unusable signal”) by using a first classification model of a pre-learned model.

The second classification unit 130 classifies the one or more segments classified as the unclear signals or unusable signals into the unclear signals and the unusable signals by using the pre-learned model.

In particular, the second classification unit 130 may classify the remaining segments other than the segment classified as the “usable signal” by the first classification unit 120 into two classes (the “unclear signal” and the “unusable signal”) by using the second classification model of the pre-learned model.

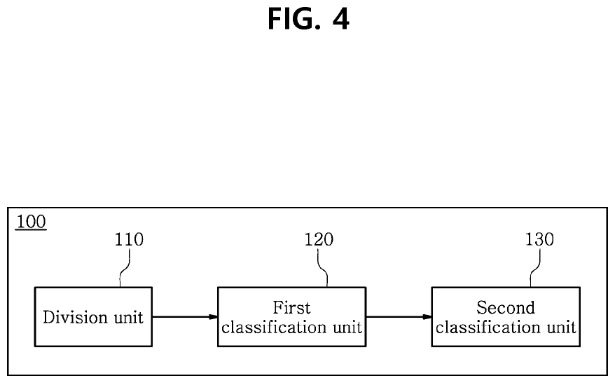

FIG. 5 is an exemplary diagram for illustrating an example in which a plurality of segments are generated.

Referring to FIG. 5, the division unit 110 divides the biosignal measured for 60 minutes in a unit time of 20 seconds into 180 segments. The division unit 110 divides the biosignal at 250 Hz into 20 segments having 5000 waveforms. In this case, the biosignal may include a plurality of different biosignals, for example, a plurality of tracks including an electrocardiogram, a photoplethysmography, an Ambulatory Blood Pressure (ABP), average waveforms of the ambulatory blood pressure, etc.

Meanwhile, the length of the biosignal and the unit time has been described as 60 minutes and 20 seconds, respectively, but they are illustrative and not necessarily limited thereto. In particular, the unit time is included as long as it is a unit of time short enough to allow a human to determine data quality at a glance, and is not necessarily limited to 20 seconds.

On the other hand, in FIG. 5, the biosignals are described as including a total of four tracks: an electrocardiogram, a photoplethysmography, an ambulatory blood pressure, an average of the ambulatory blood pressure, but they are illustrative, and the number and types of biosignals measured are not limited thereto.

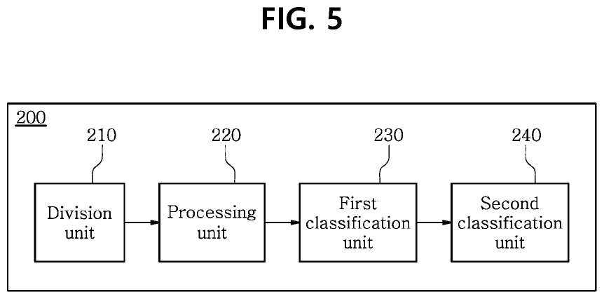

FIG. 6 is a block diagram for illustrating an apparatus 200 for determining the quality of biosignal according to an additional embodiment.

Referring to FIG. 6, the apparatus 100 for determining the quality of biosignal according to an additional embodiment may further include a processing unit 220.

In the example illustrated in FIG. 6, the division unit 210, the first classification unit 230, and the second classification unit 240 have the same configuration as that shown in FIG. 4, and thus redundant description thereof will be omitted.

Meanwhile, the processing unit 220 may be implemented by using one or more physically separated devices, or may be implemented by one or more processors or a combination of one or more processor and software, and may not be clearly distinguished in specific operations, unlike the illustrated example.

The processing unit 220 may process a Fast Fourier Transform (FFT) on biosignal to extract a feature used for learning. In this case, the biosignal to be subjected to the FFT may be preferably a photoplethysmography, but the biosignal to be subjected to the FFT processing may be various biosignals including an electrocardiogram.

As another example, the processing unit 220 may perform FFT processing on the biosignal to identify a maximum point, a minimum point, a maximum interval, a minimum interval, an amplitude, and the like of the signal on the transformed time domain. Further, the processing unit 220 may also process the calculation using the identified values.

As another example, the processing unit 220 may remove various noises including baseline sway in a preprocessing process. Specifically, the processing unit 220 may remove the baseline sway by using at least one of a preset package, library, and module (for example, a python package HeartPy).

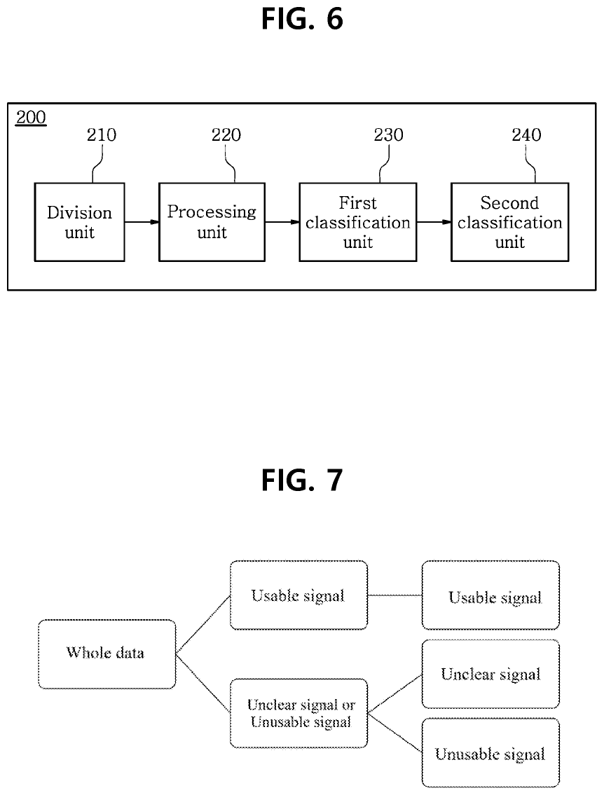

FIG. 7 is an exemplary diagram for illustrating an example of a pre-learned model.

Referring to FIG. 7, the pre-learned model may be a 2-step classifier using a Convolution Neural Network (CNN).

Meanwhile, the convolution neural network may preferably be a 1D-CNN to handle the one-dimensional waveform data, but depending on the format of the biosignal, the convolution neural network may be a 2D-CNN and/or a 3D-CNN, and is not necessarily limited to the 1D-CN.

On the other hand, the model is an algorithm for classification learning, and may be learned based on all algorithms used in machine learning, deep learning. For example, the model may be learned based on Deep Learning, random forest, Naive Bayes, support vector machine (SVM), and/or Logistic Regression. In addition, CNN-based VGGNets and/or ResNet may be used for the model.

The first classification unit 120 may classify signals into “usable signal” and other signals by using a first classification model included in the pre-learned model in the first step. The second classification unit 130 may classify other signals into an “unclear signal” and an “unusable signal” by using a second classification model included in a pre-learned model in the second step.

Specifically, the first classification model may be learned to classify the segments of the biosignals input to the first classification model into the usable signals and the unclear signals or unusable signals, based on learning data labeled with the usable class and the unusable class. In other words, the first classification model may be learned except for the learning data labeled with the unclear class.

The second classification model may be learned to classify the segments of the biosignals input to the second classification model into unclear signals and unusable signals, based on the learning data labeled with the usable class, the obscured class, and the unusable class.

The first classification model may be learned to, when a first feature of the segments of biosignal input to the first classification model is equal to or greater than a first numerical value, classify the segment of input biosignal into the unclear signals or unusable signals, and, when it is less than the first numerical value, classify it into the usable signals. On the other hand, an arrhythmia signal that is not fatal and does not affect signal interpretation is considered as normal signal.

The second classification model may be learned to, by using the second classification model, when a first feature of the segments of biosignal input to the second classification model is equal to or greater than a second numerical value having a value higher than the first numerical value, classify the input biosignal into the unusable signals, and, when it is equal to or greater than the first numerical value and less than the second numerical value, classify it into the unclear signals.

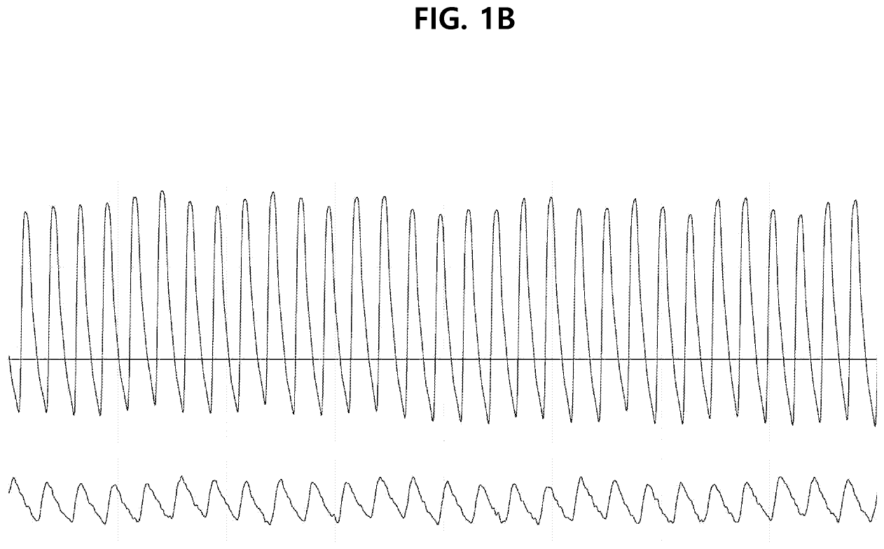

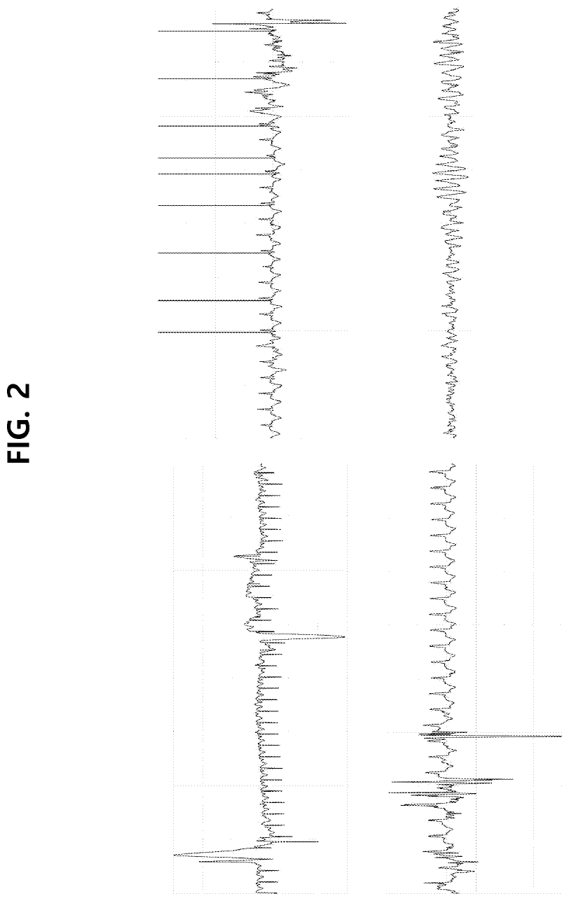

In this case, the first feature may include a feature regarding a proportion of abnormal records containing noise. Specifically, the types of noise include, but are not necessarily limited to, various noises such as baseline sway, pacemaker signal, motion artifacts, electromyogram (EMG), instrumentation noise, etc., as shown in FIG. 2 and FIG. 3.

For example, when the noise ratio of the segments of the biosignals input to the first classification model using the first classification model is 5% or more of the total, the first classification unit 120 classifies the segments into the “unclear signal or unusable signal”, and when the noise ratio is less than 5% of the total, classifies the segments into the “usable signal”. When the noise ratio of the segments of the biosignals input to the second classification model is 5% or more and less than 10% of the total, the second classification unit 130 may classify the segments into the “unclear signal”, and when it is 10% or more, the segments may be classified as the “unusable signal”.

Alternatively, the second classification model may be learned to, when the segments of the biosignals input to the second classification model contain a signal interruption and/or a fatal defect, classify the segments into the “unusable signal”. In this case, the second classification model may be learned to, when the segments contain a signal interruption and/or a fatal defect, classify the segments into the “unusable signal” irrespective of the proportion of abnormal records containing noise. Alternatively, the second classification model may be learned to, only if the first feature of the segments of the input biosignals is equal to or greater than a second numerical value, and the segments contain a signal interruption and/or a fatal defect, classify the segments into the “unusable signal”.

Here, the fatal defect may include, for example, a case where a value of a peak is not identified or a waveform of a specific portion includes a portion that is larger or smaller than an amplitude of an existing waveform by a preset proportion or more.

Further, the first classification model and the second classification model may be learned to classify the quality of the segments of the input biosignals, based on the second feature, the third feature, the fourth feature extracted from the biosignals in addition to the first feature.

Here, the second feature may include at least one of a slope between a maximum point and a minimum point in a time domain of input biosignal, an amplitude of a peak, a time interval between peaks, a number of peaks for each of sections of the segments of input biosignal, an amplitude of maximum amplitude peak for each of the sections, an amplitude of minimum amplitude peak for each of the sections, a maximum value of peak interval for each of the sections, a minimum value of peak interval for each of the sections, and spectral entropy for each of the sections.

The third feature may include at least one of an average, a standard deviation value, of each of second features, and a number of instances that deviate from the range defined by the average plus or minus the standard deviation.

The fourth feature may be based on a maximum power frequency on a time domain for each section of segments of input biosignal by performing FFT processing on each of the sections. Specifically, the fourth feature may include at least one of a standard deviation obtained by calculating for each section an average of maximum frequency difference values for each sub-section of each of the sections, an average maximum power frequency for each of the sections, and an average and a standard deviation of a differences between a first maximum power frequency and a second maximum power frequency for each of the sections.

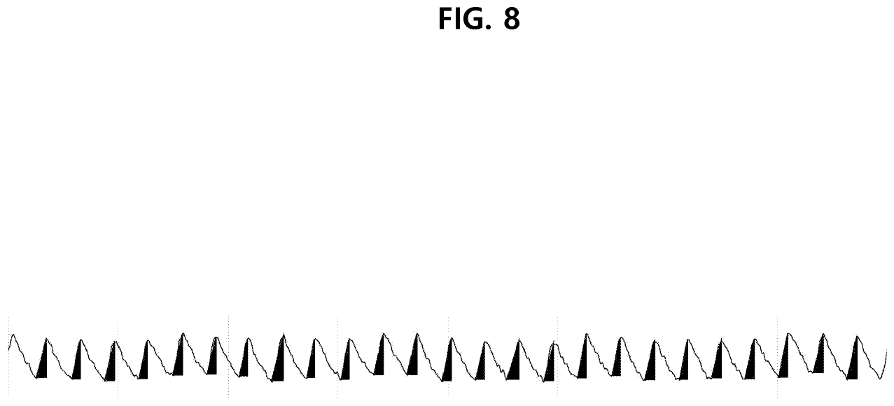

FIGS. 8 to 16 are exemplary diagrams for illustrating the second feature.

Referring to FIG. 8, the slope of the electrocardiogram is shown. The slope may mean a slope between adjacent minimum point and maximum point. The slope may be calculated from minimum point or maximum point identified from when the bit of the electrocardiogram begins. Alternatively, the slope can be calculated from the minimum or maximum point after a specific time period in the electrocardiogram.

In this case, the third feature may include at least one of the average (mean(slope)), the standard deviation (stddev(slope)) of the slopes, and the number of slopes out of the average±standard deviation range (sum(howmany(slopestotal<mean(slopestotal)−stddev(slopestotal)), howmany (slopestotal>mean(slopestotal)+stddev(slopestotal))) among the obtained slopes.

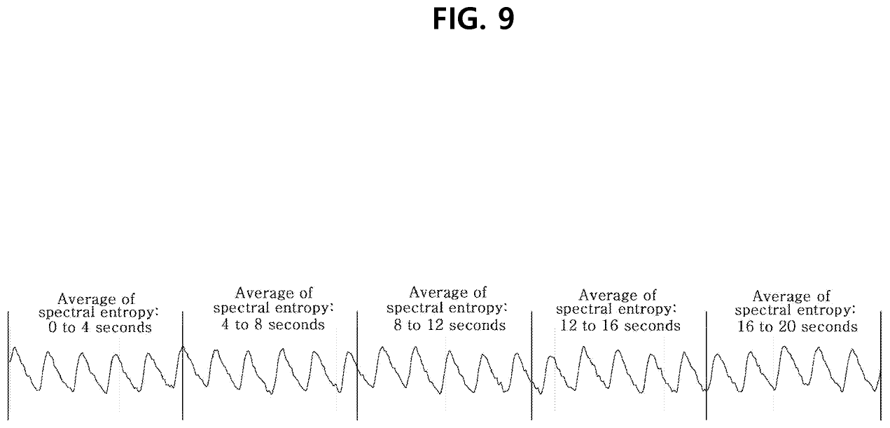

Referring to FIG. 9, the processing unit 220 calculates a spectrum for each section. As shown in FIG. 9, first, the division unit 110 divides the segments into five sections for four seconds each. Then, the processing unit 220 may calculate the spectral entropy for each section.

In this case, the third feature may include at least one of a average (mean(entropy_{spectral_4s})) and a standard deviation (stddev(entropy_{spectral_4s})) of the spectral entropy.

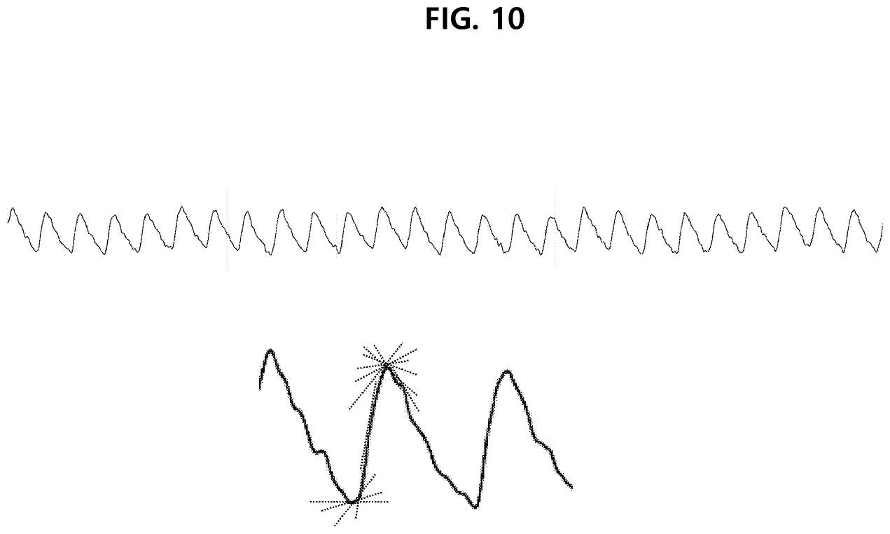

Referring to FIG. 10, the first derivative coefficient of the waveform included in the segment is shown. As shown in FIG. 10, first, the division unit 110 divides a segment of 20 seconds into five sections of 4 seconds each. Then, the processing unit 220 may calculate 4,998 first derivative coefficients from 5000 waveforms per segment for the biosignal at 250 Hz.

The third feature may include at least one of a average (mean(f′(signal))) and a standard deviation (stddev(f′(signal))) of the first derivative coefficients.

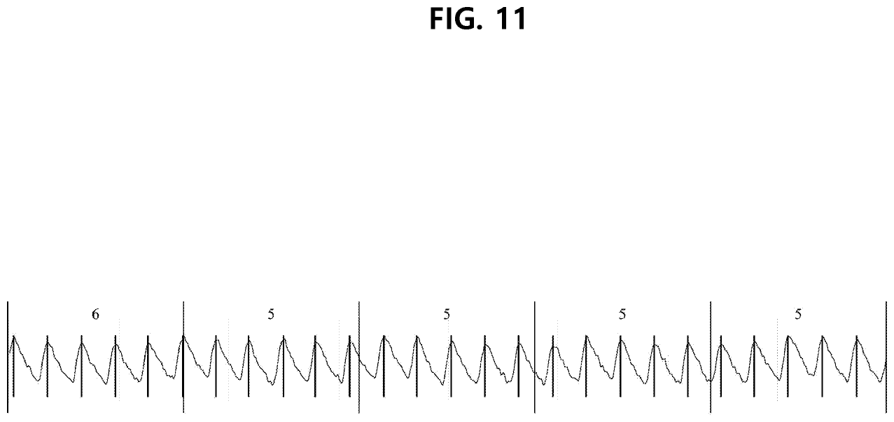

Referring to FIG. 11, the number of peaks for each section of segments is shown. As shown in FIG. 11, first, the division unit 110 divides the 20-second segments of into 5 sections by 4 seconds. Thereafter, the processing unit 220 identifies the number of peaks (6, 5, 5, 5 and 5) in each section. In this case, the third feature may include at least one of an average (mean(peaks₄ₛ)), a standard deviation (stddev(peaks₄ₛ)) of the number of peaks of each section, and the number of sections out of the average±standard deviation range (sum(howmany(peakstotal<mean(peaks₄ₛ)−stddev(peaks₄ₛ)), howmany(peakstotal>mean(peaks₄ₛ)+stddev(peaks₄ₛ)))) among the obtained number of peaks.

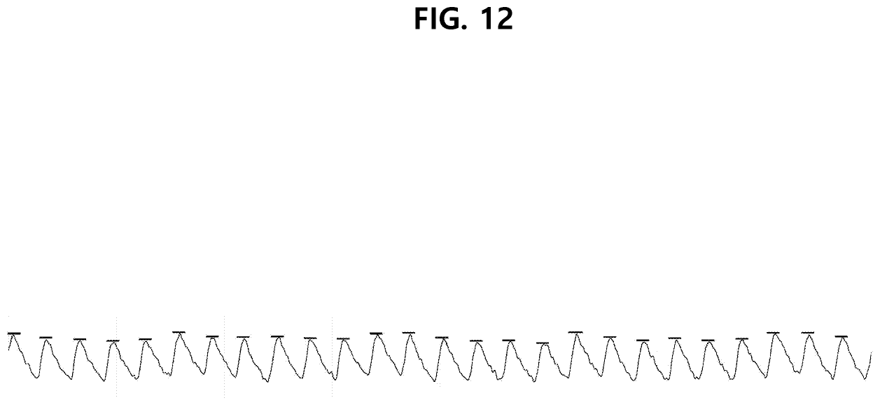

Referring to FIG. 12, the amplitude of the peak for each section of segments is shown. First, the division unit 110 divides the 20-second segments of into 5 sections by 4 seconds. Then, the processing unit 220 identifies the magnitude of the peak amplitude of each section. In this case, the third feature may include at least one of an average (mean(peakamplitudesₜₒₜₐₗ)) and a standard deviation (stddev(peakamplitudesₜₒₜₐₗ)) of the amplitudes of the peaks, and the number of those out of the range of the mean standard deviation (sum(howmany(peakamplitude<mean(peakamplitudesₜₒₜₐₗ)−stddev(peakamplitudesₜₒₜₐₗ)), howmany(peakamplitude>mean(peakamplitudesₜₒₜₐₗ)+stddev (peakamplitudesₜₒₜₐₗ)))) among the obtained amplitudes of the peaks.

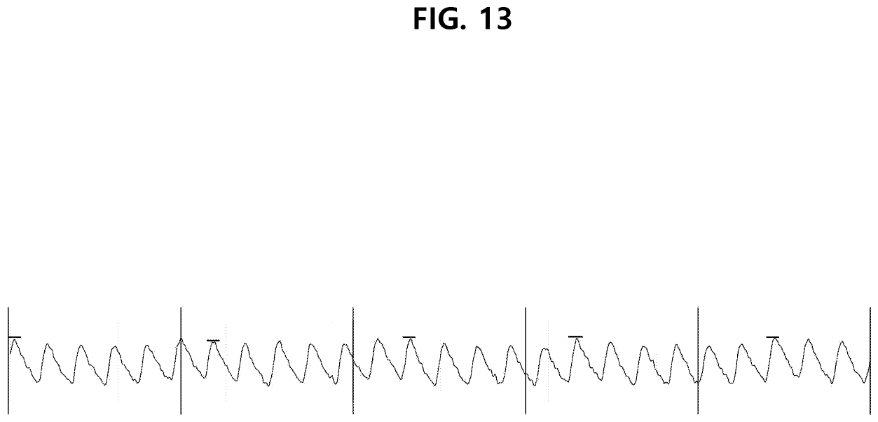

Referring to FIG. 13, the amplitude of the maximum amplitude peak for each section of segments is shown. First, the division unit 110 divides the 20-second segments of into 5 sections by 4 seconds. Then, the processing unit 220 identifies the amplitude of the peak having the maximum amplitude for each section. In the case of FIG. 13, the processing unit 220 selects a value of an amplitude of a first peak for a first section, a value of a second peak for a second section, an amplitude value of the second peak for a third section, a value of an amplitude of the second peaks for a fourth section, and a value of a third peak for a fifth section.

The third feature may include at least one of an average (mean(maxpeakamplitudes₄ₛ)), a standard deviation value (stddev(maxpeakamplitudes₄ₛ)) of the amplitudes of the maximum amplitude peaks for each selected section, and the number of those out of the average±standard deviation range (sum(howmany(maxpeakamplitudes₄ₛ<mean(maxpeakamplitudes₄ₛ)−stddev(maxpeakamplitudes₄ₛ)), howmany(maxpeakamplitudes₄ₛ>mean(maxakamplitudes₄ₛ)+stddev(maxpeakamplitudes₄ₛ))) among the obtained amplitudes of the maximum amplitude peaks for each section.

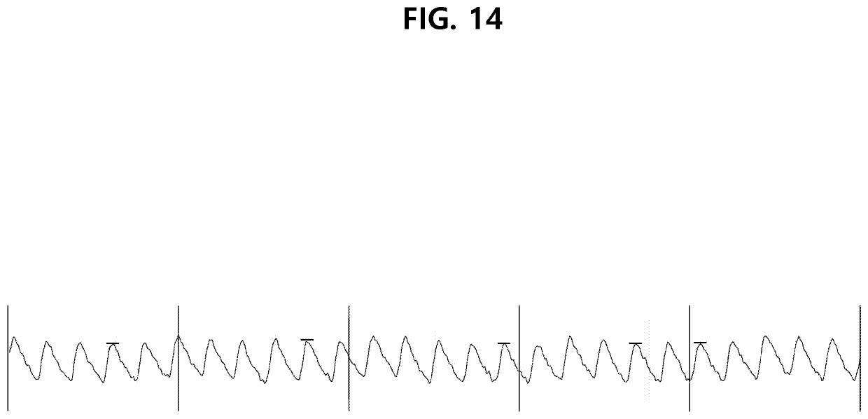

Referring to FIG. 14, the amplitude of the minimum amplitude peak for each section of segments is shown. First, the division unit 110 divides the 20-second segments of into 5 sections by 4 seconds. Then, the processing unit 220 identifies the amplitude of the peak having the minimum amplitude for each section. In the case of FIG. 14, the processing unit 220 selects a value of an amplitude of a fourth peak for a first section, a value of a fifth peak for a second section, an amplitude value of the fifth peak for a third section, a value of an amplitude of the fourth peaks for a fourth section, and a value of a first peak for a fifth section.

The third feature may include at least one of an average (mean(minpeakamplitudes₄ₛ)), a standard deviation value (stddev(minpeakamplitudes₄ₛ)) of the amplitudes of the minimum amplitude peaks for each selected section, and the number of those out of the average±standard deviation range (sum(howmany(minpeakamplitudes₄ₛ<mean(minpeakamplitudes₄ₛ)−stddev(minpeakamplitudes₄ₛ)), howmany(minpeakamplitudes₄ₛ>mean(minakamplitudes₄ₛ)+stddev(minpeakamplitudes₄ₛ)) among the obtained amplitudes of the minimum amplitude peaks for each section.

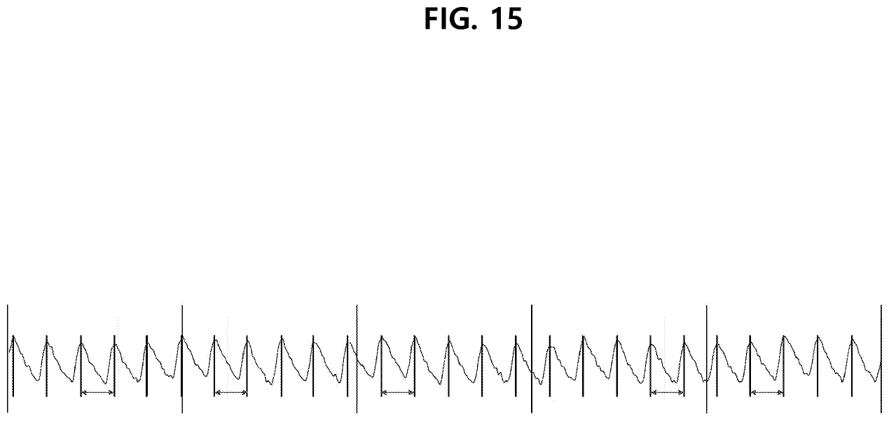

Referring to FIG. 15, the minimum value of the peak interval for each section is shown. As shown in FIG. 15, a peak interval for each section may be determined based on a peak of a signal. On the other hand, the reference of the interval is illustrative, and is not necessarily limited thereto.

First, the division unit 110 divides the 20-second segments of into 5 sections by 4 seconds. Then, the processing unit 220 identifies the peak interval for each section. In the case of FIG. 15, the processing unit 220 selects a third peak interval for a first section, a second peak interval for a second section, a second peak interval for a third section, a fifth peak interval for a fourth section, and a third peak interval for a fifth section.

The third feature may include at least one of an average (mean(minpeakintervals₄ₛ)), a standard deviation value (stddev(minpeakintervals₄ₛ)) of minimum peak intervals for each selected section, and the number of those out of the average±standard deviation range (sum(howmany(minpeakintervals₄ₛ<mean(minpeakintervals₄ₛ)−stddev(minpeakintervals₄ₛ)), howmany (minpeakintervals₄ₛ>mean(minakamplitudes₄ₛ)+stddev(minpeakintervals₄ₛ))) among the obtained amplitudes of the minimum amplitude peaks for each section.

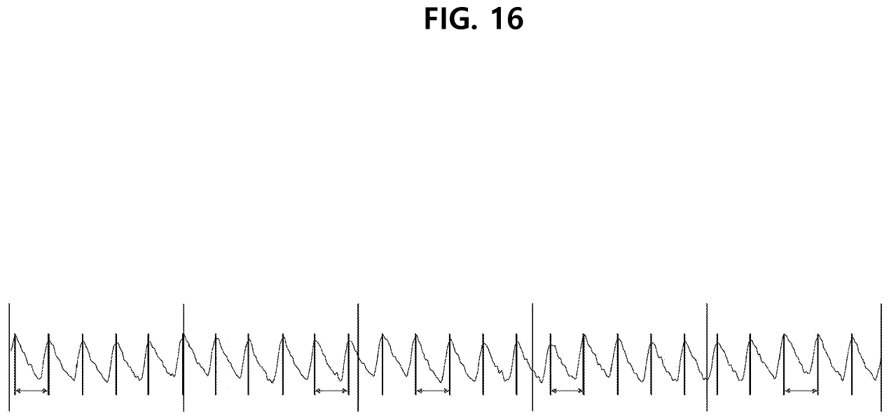

Referring to FIG. 16, the maximum value of the peak interval for each section is shown. First, the division unit 110 divides the 20-second segments of into 5 sections by 4 seconds. Then, the processing unit 220 identifies the peak interval for each section. In the case of FIG. 16, the processing unit 220 selects a second peak interval for a first section, a fifth peak interval for a second section, a third peak interval for a third section, a second peak interval for a fourth section, and a fourth peak interval for a fifth section.

The third feature may include at least one of an average (mean(maxpeakintervals₄ₛ)), a standard deviation value (stddev(maxpeakintervals₄ₛ)) of the maximum peak intervals for each selected section, and the number of those out of the average±standard deviation range (sum(howmany(maxpeakintervals₄ₛ<mean(maxpeakintervals₄ₛ)−stddev(maxpeakintervals₄ₛ)), howmany(maxpeakintervals₄ₛ>mean(maxakamplitudes₄ₛ)+stddev(maxpeakintervals₄ₛ) among the obtained amplitudes of the maximum amplitude peaks for each section.

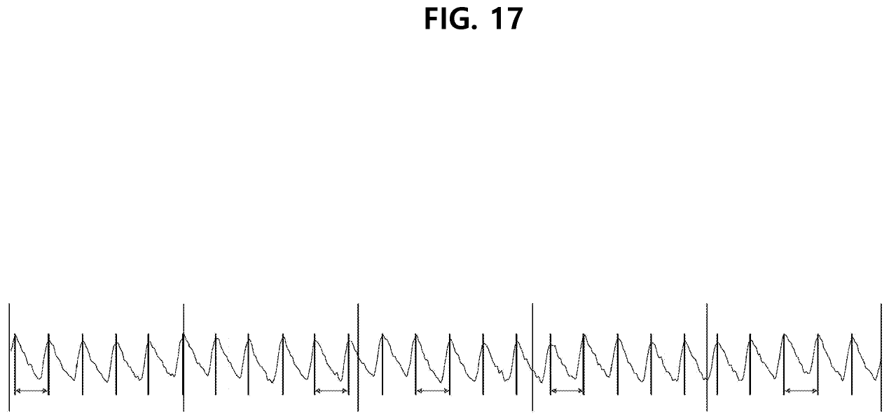

FIG. 17 is an exemplary diagram for illustrating an standard deviation obtained by calculating an average of maximum frequency difference values for each sub-section among features used by a pre-learned model for learning.

Referring to FIG. 17, first, the division unit 110 divides the segments into a plurality of sections having a constant length. For example, the division unit 110 divides the 20-second segments into 5 sections (0 to 4 seconds, 4 to 8 seconds, 8 to 12 seconds, 12 to 16 seconds, and 16 to 20 seconds) by 4 seconds. The processing unit 220 performs Fast Fourier Transform (FFT) processing on each divided section. Then, the division unit 110 divides each FFT-processed section into 8 sub-sections of 0 to 1 Hz, 1 to 2 Hz, 2 to 3 Hz, . . . and 7 to 8 Hz. At this time, the processing unit 220 identifies the maximum power frequency in each subinterval. Then, the processing unit 220 calculates an average of the difference between the maximum power frequencies for each section. Thereafter, the processing unit 220 calculates the standard deviation stddev (stddev(mean(diff(maxfreq4s)))) of the averages for each section.

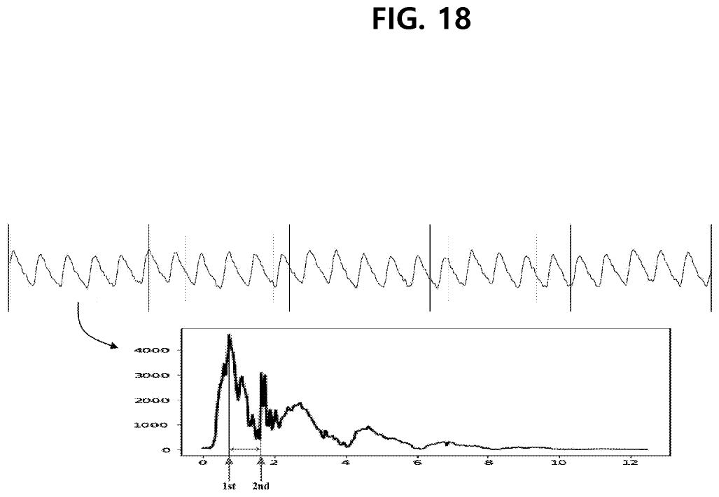

FIG. 18 is an exemplary diagram for illustrating an average and standard deviation of a difference between a first maximum power frequency and a second maximum power frequency for each section among features used by a pre-learned model for learning.

Referring to FIG. 18, first, the division unit 110 divides the segments into a plurality of sections having a constant length. For example, the division unit 110 divides the 20-second segments of into 5 sections (0 to 4 seconds, 4 to 8 seconds, 8 to 12 seconds, 12 to 16 seconds, and 16 to 20 seconds) by 4 seconds. Then, the processing unit 220 performs an FFT process on each section. Then, the processing unit 220 calculates a difference between the first maximum power frequency and the second maximum power frequency for each section. Thereafter, the processing unit 220 calculates an average (mean(diff(maxpower, secondmaxpower)₄ₛ)) and/or a standard deviation (stddev(diff(maxpower, secondmaxpower)₄ₛ)) of the difference for each section.

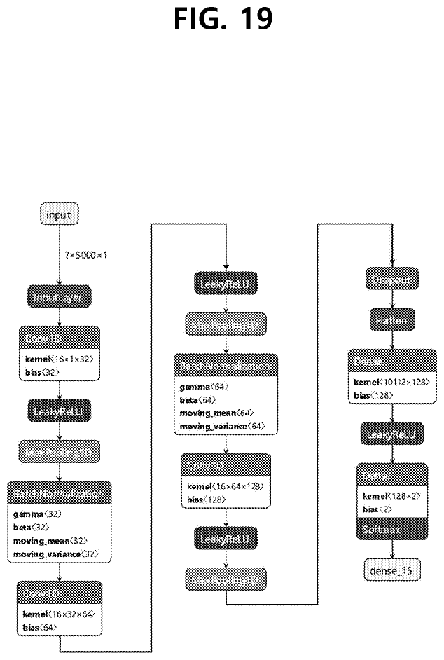

FIG. 19 is an exemplary diagram for illustrating architecture of an example of a model.

Referring to FIG. 19, the model is a CNN-based learning model, and may be designed based on a convolution layer, an activation function, a pooling layer, a normalization layer, and a drop-out.

Meanwhile, parameters such as the kernel size and bias, described in FIG. 19 are illustrative, and are not necessarily limited thereto.

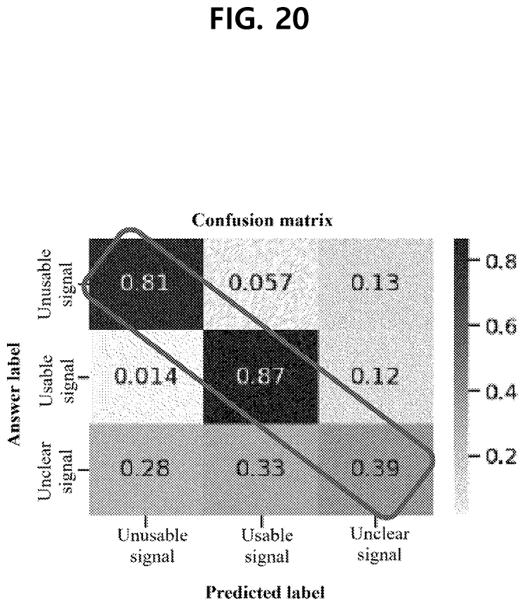

FIG. 20 is a graph showing the performance of a model having the architecture of FIG. 19 learned using the features used in FIGS. 8-18.

Referring to FIG. 20, the model has an accuracy of 0.93 as usable signals (inc) for a photoplethysmography. The model has an accuracy of 0.74 as the unusable signals (exc) for the photoplethysmography. The model has an accuracy of 0.33 as the unclear signals (uncertain) for the photoplethysmography.

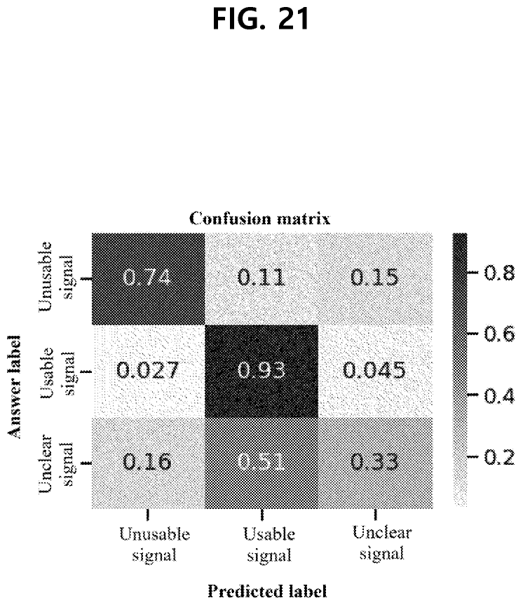

FIG. 21 is a graph showing performance of a model having another architecture.

Referring to FIG. 21, a graph shows the performance of a model designed using ResNet18. The model has an accuracy of 0.95 as the usable signals (inc) for the photoplethysmography. The model has an accuracy of 0.92 as the unusable signals (exc) for the photoplethysmography. The model has an accuracy of 0.46 as the unclear signals (uncertain) for the photoplethysmography. That is, the model using ResNet18 may have improved accuracy compared to the model of FIG. 19.

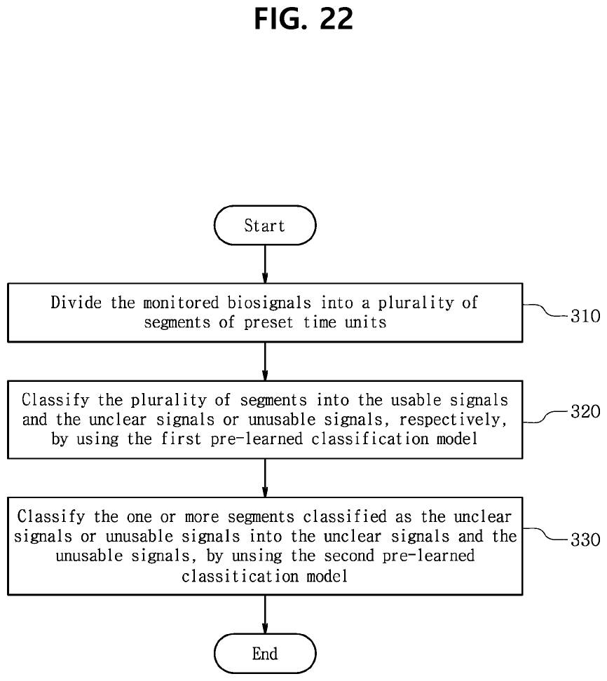

FIG. 22 is a flowchart for illustrating a method for determining the quality of biosignal according to an embodiment.

The method shown in FIG. 22 may be performed by the apparatus 100 for determining the quality of biosignal according to an embodiment of FIG. 4.

Referring to FIG. 22, the apparatus 100 for determining the quality of biosignal according to an embodiment divides the monitored biosignals into a plurality of segments of preset time units (310).

Then, the apparatus 100 for determining the quality of biosignal according to an embodiment classifies the plurality of segments into the usable signals and the unclear signals or unusable signals, respectively, by using the pre-learned model including the first classification model and the second classification model (320). Here, the unclear signal or unusable signal is classified with respect to the remaining segments except for the segment classified as the usable signals among the plurality of segments.

Then, the apparatus 100 for determining the quality of biosignal according to an embodiment classifies the one or more segments classified as the unclear signals or unusable signals into the unclear signals and the unusable signals by using the pre-learned model (330).

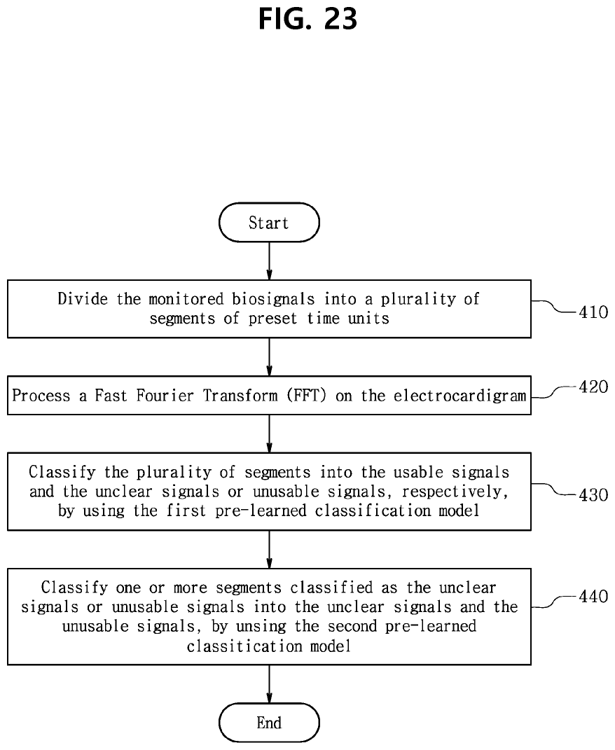

FIG. 23 is a flowchart for illustrating a method for determining the quality of biosignal according to an additional embodiment.

The method shown in FIG. 23 may be performed by the apparatus 200 for determining the quality of biosignal of FIG. 6.

Referring to FIG. 23, the apparatus 200 for determining the quality of biosignal according to an additional embodiment divides the monitored biosignals into a plurality of segments of preset time units (410).

Then, when the biosignal is an electrocardiogram, the apparatus 200 for determining the quality of biosignal according to an additional embodiment processes a Fast Fourier Transform (FFT) on the electrocardiogram (420).

Then, the apparatus 200 for determining the quality of biosignal according to an additional embodiment classifies the plurality of segments into the usable signals and the unclear signals or unusable signals, respectively, by using the pre-learned model including the first classification model and the second classification model (430). Here, the unclear signal or unusable signal is classified with respect to the remaining segments except for the segment classified as the usable signal among the plurality of segments.

Thereafter, the apparatus 200 for determining the quality of biosignal according to an additional embodiment classifies one or more segments classified as the unclear signals or unusable signals into the unclear signals and the unusable signals by using a pre-learned model (440).

In the FIGS. 22 and 23 shown above, the method has been described with reference to the flowchart shown in the drawings. Although, for purposes of explanation, the method is shown and described as a series of blocks, the present disclosure is not limited by the order of the blocks, as some blocks may occur in different orders or concurrently with other blocks from that shown and described herein, and various other branches, flow paths, and orders of blocks may be implemented which achieve the same or similar results. Moreover, not all shown blocks may be required for implementation of the methods described herein.

Furthermore, the method according to an embodiment of the present disclosure may be implemented in the form of a computer program for performing a series of processes, and the computer program may be recorded on a computer-readable recording medium. Examples of the computer-readable recording medium include magnetic media such as a hard disk, a floppy disk, and a magnetic tape, optical recording media such as a CD-ROM and a DVD, magneto-optical media such as a floptical disk, and hardware devices specially configured to store and perform program instructions such as a ROM, a RAM, and a flash memory.

While the foregoing has been described with reference to embodiments, it will be understood by those skilled in the art that various modifications and variations can be made to the disclosure without departing from the spirit and scope of the disclosure as described in the following claims.

### INDUSTRIAL APPLICABILITY

The apparatus and method for determining the quality of biosignal data according to an embodiment are applicable to the digital medical industry by automatically determining the quality of biosignal without human intervention using an algorithm.

1. A method for determining the quality of biosignal data, performed by an apparatus for determining the quality of biosignal data comprising:
one or more processors; and
a memory storing one or more programs executed by the one or more processors,
the method comprising:
dividing a monitored biosignal into a plurality of segments of preset time units;
classifying the plurality of segments into usable signals and unclear signals or unusable signals, respectively, by using a pre-learned model comprising a first classification model and a second classification model, wherein the unclear signals or unusable signals are classified for remaining segments except for segments classified as the usable signals among the plurality of segments; and
classifying, by using the pre-learned model, one or more segments that are classified as the unclear signals or unusable signals into the unclear signals and the unusable signals.

2. The method for determining the quality of biosignal data of claim 1, wherein
the biosignal comprises at least one of an electrocardiogram and a photoplethysmography, and
the method further comprises, when the biosignal is an electrocardiogram, processing a Fast Fourier Transform (FFT) on the electrocardiogram.

3. The method for determining the quality of biosignal data of claim 2, wherein
the first classification model is learned to classify the segments of biosignal input to the first classification model into usable signals and unclear signals or unusable signals, based on learning data labeled with an usable class and an unusable class, and
the second classification model is learned to classify the segments for biosignal input to the second classification model into unclear signals and unusable signals, based on learning data labeled with an usable class, an unclear class, and an unusable class.

4. The method for determining the quality of biosignal data of claim 3, wherein
the first classification model is learned to, when a first feature of the segments of biosignal input to the first classification model is equal to or greater than a first numerical value, classify the segment of input biosignal into the unclear signals or unusable signals, and, when it is less than the first numerical value, classify it into the usable signals,
the second classification model is learned to, when a first feature of the segments of biosignal input to the second classification model is equal to or greater than a second numerical value having a value higher than the first numerical value, classify the input biosignal into the unusable signals, and, when it is equal to or greater than the first numerical value and less than the second numerical value, classify it into the unclear signals, and, when it is less than the first numerical values, classify it into the usable signals, and
first feature comprises a proportion of abnormal records containing noise or signal interruption in the segments of input biosignal.

5. The method for determining the quality of biosignal data of claim 3, wherein
the first classification model is learned to classify the segments of input biosignal into the usable signals and the unclear signals or unusable signals, based on second feature of the segments of biosignal input to the first classification model,
the second classification model is learned to classify the segments of input biosignal into the usable signals, the unclear signals, and the unusable signals, based on second feature of the segments of biosignal input to the second classification model, and
second feature comprises at least one of a slope between a maximum point and a minimum point in a time domain of input biosignal, an amplitude of a peak, a time interval between peaks, a number of peaks for each of sections of the segments of input biosignal, an amplitude of maximum amplitude peak for each of the sections, an amplitude of minimum amplitude peak for each of the sections, a maximum value of peak interval for each of the sections, a minimum value of peak interval for each of the sections, and spectral entropy for each of the sections.

6. The method for determining the quality of biosignal data of claim 5, wherein
the first classification model is learned to classify the segments of input biosignal into the usable signals and the unclear signals or unusable signals, based on third feature of the segments of biosignal input to the first classification model,
the second classification model is learned to classify the segments of input biosignal into the usable signals, the unclear signals, and the unusable signals, based on third feature of the segments of biosignal input to the second classification model, and
third feature comprises at least one of an average, a standard deviation value, of each of second features, and a number of instances that deviate from the range defined by the average plus or minus the standard deviation.

7. The method for determining the quality of biosignal data of claim 3, wherein
the first classification model is learned to classify the segments of the input biosignal into the usable signals and the unclear signals or unusable signals, based on fourth feature of the segments of biosignal input to the first classification model,
the second classification model is learned to classify the segments of input biosignal into the usable signals, the unclear signals, and the unusable signals, based on fourth feature of the segments of biosignal input to the second classification model, and
fourth feature is based on a maximum power frequency on a time domain for each section of segments of input biosignal by performing FFT processing on each of the sections.

8. The method for determining the quality of biosignal data of claim 7, wherein
the fourth feature comprises at least one of a standard deviation obtained by calculating for each section an average of maximum frequency difference values for each sub-section of each of the sections, an average and a standard deviation of the average maximum power frequencies for each of the sections, and an average and a standard deviation of a differences between a first maximum power frequency and a second maximum power frequency for each of the sections.

9. An apparatus for determining the quality of biosignal data, comprising:
one or more processors; and
a memory storing one or more programs executed by the one or more processors,
wherein the one or more processors:
divide a monitored biosignal into a plurality of segments of preset time units;
classify the plurality of segments into usable signals and unclear signals or unusable signals, respectively, by using a pre-learned model comprising a first classification model and a second classification model, wherein the unclear signals or unusable signals are classified for remaining segments except for segments classified as the usable signals among the plurality of segments, and
classify, by using the pre-learned model, one or more segments that are classified as the unclear signals or unusable signals into the unclear signals and the unusable signals.

10. The apparatus for determining the quality of biosignal data of claim 9, wherein
the biosignal comprises at least one of an electrocardiogram and a photoplethysmography, and
the one or more processors:
when the biosignal is an electrocardiogram, process a Fast Fourier Transform (FFT) on the electrocardiogram.

11. The apparatus for determining the quality of biosignal data of claim 10, wherein
the first classification model is learned to classify the segments of biosignal input to the first classification model into usable signals and unclear signals or unusable signals, based on learning data labeled with an usable class and an unusable class, and
the second classification model is learned to classify the segments for biosignal input to the second classification model into unclear signals and unusable signals, based on learning data labeled with an usable class, an unclear class, and an unusable class.

12. The apparatus for determining the quality of biosignal data of claim 11, wherein
the first classification model is learned to, when a first feature of the segments of biosignal input to the first classification model is equal to or greater than a first numerical value, classify the segment of input biosignal into the unclear signals or unusable signals, and, when it is less than the first numerical value, classify it into the usable signals,
the second classification model is learned to, when a first feature of the segments of biosignal input to the second classification model is equal to or greater than a second numerical value having a value higher than the first numerical value, classify the input biosignal into the unusable signals, and, when it is equal to or greater than the first numerical value and less than the second numerical value, classify it into the unclear signals, and, when it is less than the first numerical values, classify it into the usable signals, and
first feature comprises a proportion of abnormal records containing noise or signal interruption in the segments of input biosignal.

13. The apparatus for determining the quality of biosignal data of claim 11, wherein
the first classification model is learned to classify the segments of input biosignal into the usable signals and the unclear signals or unusable signals, based on second feature of the segments of biosignal input to the first classification model,
the second classification model is learned to classify the segments of input biosignal into the usable signals, the unclear signals, and the unusable signals, based on second feature of the segments of biosignal input to the second classification model, and
second feature comprises at least one of a slope between a maximum point and a minimum point in a time domain of input biosignal, an amplitude of a peak, a time interval between peaks, a number of peaks for each of sections of the segments of input biosignal, an amplitude of maximum amplitude peak for each of the sections, an amplitude of minimum amplitude peak for each of the sections, a maximum value of peak interval for each of the sections, a minimum value of peak interval for each of the sections, and spectral entropy for each of the sections.

14. The apparatus for determining the quality of biosignal data of claim 13, wherein
the first classification model is learned to classify the segments of input biosignal into the usable signals and the unclear signals or unusable signals, based on third feature of the segments of biosignal input to the first classification model,
the second classification model is learned to classify the segments of input biosignal into the usable signals, the unclear signals, and the unusable signals, based on third feature of the segments of biosignal input to the second classification model, and
third feature comprises at least one of an average, a standard deviation value, of each of second features, and a number of instances that deviate from the range defined by the average plus or minus the standard deviation.

15. The apparatus for determining the quality of biosignal data of claim 11, wherein
the first classification model is learned to classify the segments of the input biosignal into the usable signals and the unclear signals or unusable signals, based on fourth feature of the segments of biosignal input to the first classification model,
the second classification model is learned to classify the segments of input biosignal into the usable signals, the unclear signals, and the unusable signals, based on fourth feature of the segments of biosignal input to the second classification model, and
fourth feature is based on a maximum power frequency on a time domain for each section of segments of input biosignal by performing FFT processing on each of the sections.

16. The apparatus for determining the quality of biosignal data of claim 15, wherein
the fourth feature comprises at least one of a standard deviation obtained by calculating for each section an average of maximum frequency difference values for each sub-section of each of the sections, an average and a standard deviation of the average maximum power frequencies for each of the sections, and an average and a standard deviation of a differences between a first maximum power frequency and a second maximum power frequency for each of the sections.
